(19) United States
(12) Patent Application Publication
(10) Pub. No.: US 2009/0104630 A1
(43) Pub. Date: Apr. 23, 2009

(54) Semi-quantitative immunochromatographic device and method for the determination of HIV/AIDS immune-status via measurement of soluble CD40 ligand/CD 154, A CD4+T cell equivalent

(76) Inventor: Paul C. Reiter, East Hampstead, NH (US)

Correspondence Address: BODNER & O'ROURKE, LLP, 425 BROADHOLLOW ROAD, SUITE 108, MELVILLE, NY, 11747, US

(21) Appl. No.: 12/288,180

(22) Filed: Oct. 17, 2008

Related U.S. Application Data
(60) Provisional application No. 60/981,110, filed on Oct. 19, 2007.

(51) Int. Cl. G01N 33/53; C12M 1/00

(52) U.S. Cl. 435/7.21; 435/287.2

(57) ABSTRACT

A semi-quantitative, immunochromatographic device for the detection of HIV/AIDS immune status CD4+ T cell equivalents, such as soluble CD40 ligand/CD 154, includes one or more support materials capable of providing lateral flow. The one or more support materials include a first area for receiving a biological sample containing a target analyte, the analyte being a CD4+ T cell equivalent, such as soluble CD40 ligand/CD 154, a second area having a movably contained detector ligand, wherein the detector ligand is capable of forming a mobile complex with the soluble CD40 ligand/CD 154, and at least one capture area having a predetermined amount of an immobile capture reagent, the immobile capture reagent capable of specifically binding to the mobile complex formed by the soluble CD40 ligand/CD 154 protein and the detector ligand and providing a visible signal.

### CROSS-REFERENCE TO RELATED APPLICATION

This application is based on U.S. Provisional Application Ser. No. 60/981,110, filed on Oct. 19, 2007, and entitled, “Semi-Quantitative Immunochromatographic Device and Method For The Determination of HIV/AIDS Immune-Status Via Measurement of Soluble CD40 Ligand/CD 154, a CD4+ T Cell Equivalent”, the disclosure of which is incorporated herein by reference and on which priority is hereby claimed.

### BACKGROUND OF THE INVENTION

1. Field of the Invention

The present invention relates to a method and apparatus for monitoring immune system status and function, in HIV/AIDS patients. More particularly, it relates to a new and improved device and method for the semi-quantitative detection, analysis and measurement of CD4+ T cells, through their surrogate marker, i.e., soluble CD40 ligand (CD 154); a protein expressed on the surfaces of CD4+T cells following activation by HIV infection. Such semi-quantitative analysis and measurement in turn can be used to; stage HIV/AIDS patients to determine their immune status and whether anti-retroviral treatment should be initiated, or changed immediately.

2. Description of the Prior Art

There is a real need for new and better methods and apparatus for assaying CD4+ T cell levels in patients suffering from the Human Immunodeficiency Virus, irrespective of age, and irrespective of resources, particularly in third world countries where resources are scarce and men, women and children are dying in staggering numbers. Human Immunodeficiency Virus (HIV) is a retrovirus, i.e., a virus that contains two single-strand linear ribonucleic acid (RNA) molecules per virion and reverse transcriptase, which together allow the virus to transcribe RNA into DNA, instead of DNA to RNA. The virus-produced DNA in turn is incorporated into the host cell's DNA strand, which then produces new RNA retroviruses. In other words, the retrovirus restructures the host cell DNA to produce more retroviruses and thereafter kills the host cells. By killing the host cells, particular the host immunity producing cells, the retrovirus renders the host extremely vulnerable and leads to ACQUIRED IMMUNODEFICIENCY SYNDROME (AIDS), a condition in humans in which the immune system begins to fail, leading to life-threatening opportunistic infections.

Infection with HIV occurs by the transfer of blood, semen, vaginal fluid, pre-ejaculate, or breast milk. Within these bodily fluids, HIV is present as both free virus particles and virus infected immune cells. The four major routes of transmission are unprotected sexual intercourse, contaminated needles, and transmission from an infected mother to her baby at birth or through breast milk.

HIV primarily infects vital cells in the human immune system such as helper T cells and more specifically CD4+ T cells, macrophages and dendritic cells. HIV infection leads to low levels of CD4+ T cells through three main mechanisms: (i) via direct viral killing of infected cells; (ii) via increased rates of apoptosis in infected cells; and (iii) via the killing of infected CD4+ T cells by CD8 cytotoxic lymphocytes that recognize infected cells. When CD4 +T cells decline below a critical level, cell-mediated immunity declines and the body becomes progressively more susceptible to opportunistic infections. If untreated, eventually most HIV-infected individuals develop AIDS and die.

HIV infection in humans is now pandemic. As of January 2006, the Joint United Nations Programme on HIV/AIDS and the World Health Organization estimated that AIDS had killed more than 25 million people since it was first recognized on Dec. 1, 1981, making it one of the most destructive pandemics in recorded history. In 2005 alone, AIDS claimed an estimated 2.4-3.3 million lives, of which more than 570,000 were children. It is estimated that about 0.6% of the world's living population is infected with HIV.

A third of these deaths are occurring in sub-Saharan Africa, retarding economic growth and increasing poverty. According to the current estimates, HIV is set to infect 90 million people in Africa alone, resulting in a minimum estimate of 18 million orphans.

There is no cure for AIDS. On the other hand, there is anti-retroviral treatment (ART), which reduces both the mortality and the morbidity of HIV infection. The treatment consists-of drugs that have to be taken every day for the rest of the patient's life, when the time is right. They work against the HIV infection itself by slowing down the replication of the HIV in the body.

It has been found that for antiretroviral treatment (ART) to be effective for a long time, it has been found that patients need to take more than one anti-retroviral drug at a time. When HIV replicates, i.e., makes new copies of itself, it often makes mistakes. This means that within any infected person there are many different strains of the HIV. Occasionally, a new strain is produced that happens to be resistant to the effect of one type of anti-retroviral drug. If the patient is not taking any other type of drug, then the resistant strain is able to replicate quickly and the benefits of the treatment are lost. Taking two or more anti-retrovirals at the same time vastly reduces the rate at which resistance develops.

However, there is a problem. The tools for the truly routine, truly inexpensive, speedy, yet effective determination of the need for and implementation of anti-retroviral treatment (ART) is just not available in any country.

It is well known that the progression of HIV/AIDS in HIV infected patients is monitored through CD4+ T cells. Specifically, the CD4+ T cell count in peripheral blood is used for: (i) assessing the degree of immune deterioration and speed of progression towards AIDS; (ii) defining, together with clinical information, decision points to initiate anti-retroviral treatment (hereinafter “ART”); (iii) deciding the time for prophylaxis of opportunistic infections; and (iv) monitoring the efficacy of treatment. Ideally, as soon as CD4 +T cells decline below internationally recognized critical levels, anti-retroviral treatment must be started immediately.

In other words, the CD4+ T cell count is a critical parameter in monitoring HIV disease. Specifically, measurements of CD4+ T cells are essential for staging HIV-infected patients, i.e. establishing the stage or level of the infection in HIV-positive patients. Once the stage or level of infection is established, the patients need for anti-retroviral medications can be ascertained, and the decision of whether anti-retroviral therapy should be implemented can be made with certainty. Once the anti-retroviral therapy is implemented, it can be monitored through the continued measurement of CD4+ T cells. Lower numbers of circulating CD4+ T cells imply a more advanced stage of HIV disease and less competent defense mechanisms.

In developed countries, CD4+ T cell counts are typically performed every three to six months for each patient using the method of flow cytometry. In fact, flow cytometry is considered the gold standard for the determination of CD4+ T cell counts. Flow cytometry uses lasers to excite fluorescent antibody probes specific for CD4 and other cell surface markers, and to distinguish one type of lymphocyte from another.

Flow cytometry is not cheap. Further, it is technically demanding, complex and costly. Instruments that are commercially available from various manufacturers are significantly expensive, anywhere from $20,000 (USD) TO $95,000 (USD). They can be run only by operators that are sufficiently trained in both the technical and biological aspects of CD4+ T cell counting. They need special dedicated laboratory space. The counting itself is complex and therefore technically demanding. It requires expensive reagents and regular maintenance if the counts are to be precise and accurate.

All these factors, including the cost of a flow cytometer, technical and operational complexity, the need for reliable electricity, and the high cost of reagents have made the treatment of HIV/AIDS patients a very expensive proposition in all countries, irrespective of whether such countries are third world, resource-poor countries or not. In industrial nations, the factors have pushed insurance costs through the roof and seriously taxed the industrial nations' resources, while in resource-poor countries, these factors have made the use of these instruments impractical and/or difficult to sustain. Thus, the urgent need for affordable and technically simple CD4 diagnostics in both rich and resource-scarce or resource-poor settings is widely recognized, albeit the need is much more pressing in resource-poor settings.

Several efforts have been made to develop alternative, affordable CD4+ T cell counting methods. Single purpose flow cytometers have been designed solely for counting CD4 cells, such as the Becton Dickinson FASCOUNT, the Partec CYFLOW, and desktop instruments from Guava and PointCare Technologies. Low-cost microbead separation of CD4 cells from other blood cells, followed by standard manual cell counting techniques using a light microscope, have also been proposed. Although the former make flow cytometry more affordable in some settings, reagent costs remain high, and the instruments remain expensive, and in most cases, technically complex. While the latter significantly lower reagent costs, as compared to flow cytometry, they are of low throughput, extremely labor intensive, and appear to be less accurate than traditional flow cytometry. Thus, these alternative counting methods do very little to alleviate the depletion of resources and the skyrocketing of insurance costs in industrial countries. Finally, in many developing countries there are few laboratories. Those that do exist are not adequately equipped. Outlying clinics must send samples for testing and wait days for the results, thus losing the opportunity to treat patients by initiating ART, due to the fact that the patients do not return for further treatment once the clinics receive the results.

Thus, the lack of tools for the truly routine, truly inexpensive, speedy, yet effective determination of the need for and implementation of anti-retroviral treatment (ART) needs to be addressed. Treatment with anti-retrovirals, where available, increases the life expectancy of people infected with HIV. Current and future treatment may allow HIV-infected individuals to achieve a life expectancy approaching that of the general public, by proper diagnosis and immediate implementation of ART.

Accordingly, there is a real need for new and better methods and apparatus for assaying CD4+ T cell levels, including methods and apparatus that will avoid the drawbacks of prior art methods and instrumentation while at the same time provide for the assaying of CD4+ T cells at a low cost, without technical and operational complexity, without expensive reagents, without electricity, in a speedy manner while the patient is still present and capable of receiving immediate anti-retroviral therapy, if necessary. Absent such new methods, HIV infection in humans will continue to be pandemic. AIDS will continue to kill both adults and children particularly in resource-poor territories. Such deaths will continue to tax humanity worldwide because they will continue to retard economic growth and increase poverty. Finally, although the need for a simplified and inexpensive assaying of CD4+ T cells count is particularly acute and pressing in resources-poor, third-world countries, such assaying can also be beneficial to industrial countries world wide, by helping to lower their ever increasing and burdensome medical and insurance costs inflicted upon them by AIDS.

### OBJECTS OF THE INVENTION

It is therefore an object of the present invention to provide a method and apparatus that will put routine immune status testing within the reach of far more HIV patients, particularly in resource-scarce or resource-poor areas.

It is a further object of the present invention to provide a method and apparatus that will provide and make available an extremely affordable and easy to use rapid diagnostic test for CD4+ T cell levels.

It is an even further object of the present invention to provide a method and apparatus for the determination of CD4+ T cell levels that can be performed without any special instrumentation and which will require no highly skilled personnel, fresh water or electricity.

It is still another object of the present invention to provide a method and apparatus for the determination of CD4+ T cell levels within 20 minutes while the patient is still present, so there is no need for call back, days later, to read test results.

Another object of the present invention is to provide a method and apparatus that will give clinicians the ability to quickly diagnose compromised immune status due to HIV while the patient is still in their office and immediately begin ART, if necessary.

It is a further object of the present invention to provide a method and apparatus for providing a cost effective means of monitoring the efficacy of ART and the patient's treatment adherence, even in the most remote of locations.

It is yet another object of the present invention to provide a method and apparatus for the determination of CD4 cell levels at a cost of $5.00 or less versus the current cost of $25-$50 (estimated cost including reagents, equipment, sample transport, etc.), and become accessible to more patients in all settings, helping to optimize ART and the allocation of limited supplies of ART drugs to the patients who need them most.

Another object of the present invention is to provide a method and apparatus that allow the determination of CD4+ T cell levels not by measuring the cells themselves but by measuring CD4+ T cell equivalents, i.e., soluble CD40 ligand, a protein expressed on the surfaces of CD4+T cells following activation by HIV infection. Such determination will become routine in all countries and provide timely access to anti-retroviral medication even in resource-poor countries.

A further object of the present invention is to provide a method and apparatus that allow the determination of CD4 cell levels without the need for lysis of such cells.

Yet another object of the present invention is to provide a method and apparatus that allow the determination of CD4 cell levels not by measuring the cells themselves, but through a surrogate marker thereof.

These objects, as well as other objects and advantages, will become apparent from the following disclosure.

### SUMMARY OF THE INVENTION

According to the present invention, there is provided a semi-quantitative, immunochromatographic device and method for the detection of HIV/AIDS immune status CD4+ T cell equivalents, i.e., soluble CD40 ligand/CD 154. It comprises one or more support materials capable of providing lateral flow, the one or more support materials having: (a) an area for receiving a biological sample containing a target analyte, said analyte being a CD4+ T cell equivalent, namely soluble CD40 ligand/CD 154; (b) an area comprising a movably contained detector ligand, wherein the detector ligand is capable of forming a mobile complex with the soluble CD40 ligand/CD 154; and c) at least one capture area comprising a predetermined amount of an immobile capture reagent, the immobile capture reagent capable of specifically bonding to the mobile complex formed by the soluble CD40 ligand/CD 154 protein and the detector ligand and providing a visible signal.

The process of using the semi-quantitative, immunochromatographic device 10 comprises the steps of placing a serum, plasma or whole blood sample at one end of the device 10; thereafter, allowing the sample to migrate via lateral flow across the device membrane 10: through an area on the device 10 having an antigen binding monoclonal or polyclonal antibody conjugated to a colloidal gold particle area, which binds to the soluble CD40 ligand/CD 154 in the sample to form a complex; and thereafter, moving the complex, via lateral flow, over a unique combination of monoclonal and/or polyclonal antibodies immobilized on the device, which in turn bind the complex to generate a pink/purple band or bands on the device 10. The remaining complex continues to migrate to a control area on the device 10, which produces an additional band to show the test has been performed correctly. Each pink or purple band appearing in the test area of the device 10 corresponds to a number of CD4+ T cell equivalents, which for example can be correlated to one of three categories (see FIGS. 3, 4, 5 and 6): Satisfactory Immune Status, e.g., about 500 plus CD4+ T cells/μl; Decreasing Immune Status, e.g., less than about 500 plus CD4+ T cells/μl but greater than about 350 CD4+ T cells/μl; or Diminished Immune Status, e.g., less than about 200 CD4+ T cells/μl. Different levels of detection can also be achieved by manipulating the concentrations of the various reagents employed.

Furthermore, in accordance with the present invention, a method of detecting and/or quantifying the presence of CD4+ T cells includes the step of detecting and/or quantifying the presence of soluble CD40 ligand/CD 154.

### BRIEF DESCRIPTION OF THE DRAWINGS

While the specification concludes with claims which particularly point out and distinctly claim the present invention, it is believed that the present invention will be better understood from the following detailed description taken in conjunction with the accompanying drawings in which like numerals represent identical elements and wherein:

FIG. 1 is a cross-sectional, side view schematic of the inventive semi-quantitative, immunochromatographic device;

FIG. 2 is a top plan view of one of the embodiments of the semi-quantitative, immunochromatographic device formed in accordance with the present invention;

FIGS. 3-6 are top plan views of various embodiments of the semi-quantitative, immunochromatographic device formed in accordance with the present invention and showing CD4+ T cell count equivalents and staging of the immune status.

LIST OF ELEMENTS AND THEIR RESPECTIVE
IDENTIFYING NUMERALS

| NO. | ELEMENT |
|---|---|
| 10 | Semi-quantitative |
| 20 | Immunochromatographic strip |
| | Support Structure |
| 22 | Top side of the Support Structure 20 |
| 24 | Bottom side of the Support Structure 20 |
| 26 | Proximate end of the Support Structure |
| 28 | Distal end of the Support Structure 20 |
| 30 | Membrane |
| 32 | First end of Membrane |
| 34 | Second end of Membrane |
| 40 | Biological sample receiving area or |
| | sample pad |
| 50 | Control line area |
| 60 | Conjugate pad/detector ligand area |
| 70 | Capture area/Test Line(s) |
| 90 | Sink Pad |

### DETAILED DESCRIPTION OF THE INVENTION

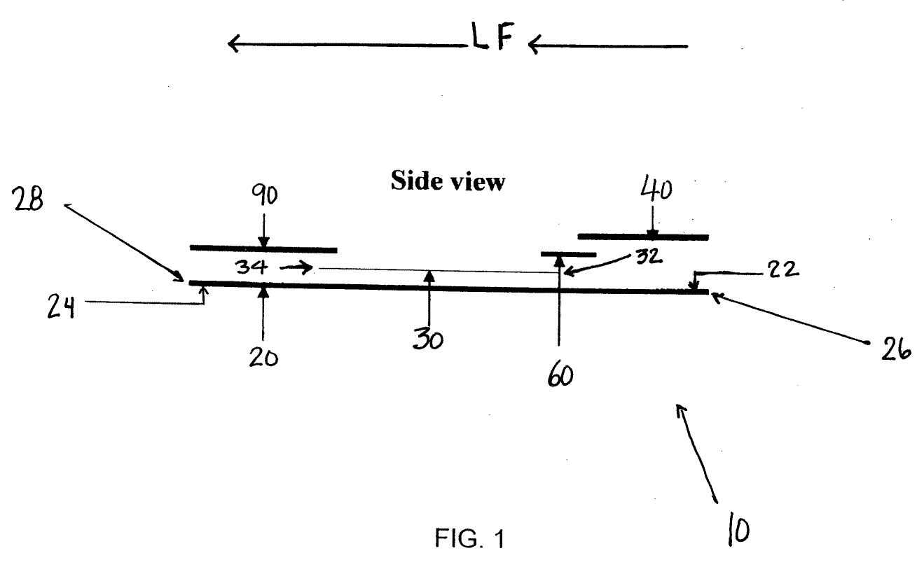

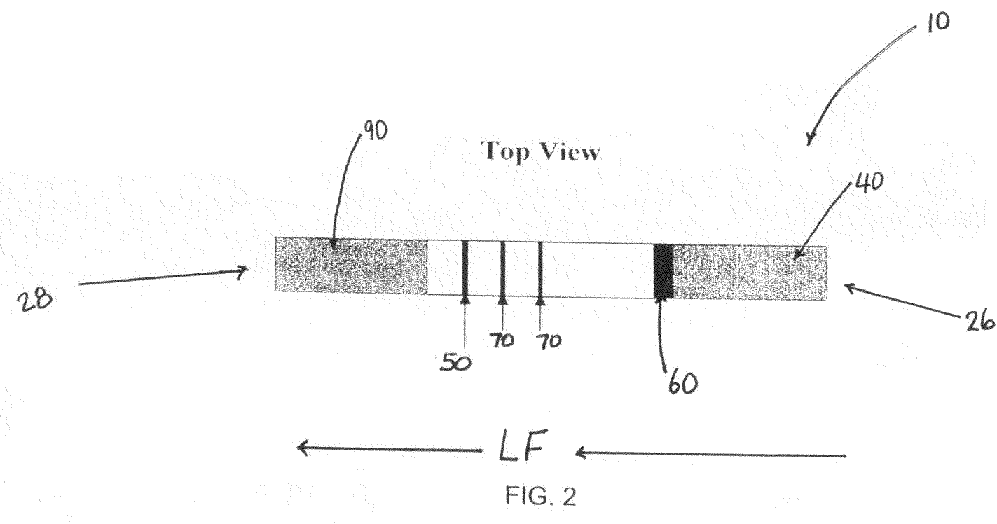

Referring specifically to the drawings, FIGS. 1 and 2 generally depict the inventive semi-quantitative, immunochromatographic test strip and method for use thereof at 10 (hereinafter “the device 10”). As has been set forth in more detail herein below, it is designed to provide an accurate semi-quantitative, membrane-based screening test for CD4+ T cell levels by assaying a CD4+ T cell equivalent Soluble CD40 ligand/CD 154. It comprises the newest generation of lateral flow immunochromatographic assay devices, which can be used on site with serum, plasma or whole blood samples.

Soluble CD40 ligand/CD 154 is a protein, which is expressed on the surfaces of CD4+ T cells following their activation by HIV infection. The serum levels of this protein correlate directly to CD4+ T cell counts. In fact, as disclosed in the article “Levels of Soluble CD40 Ligand (CD154) in Serum Are Increased in Human Immunodeficiency Virus Type 1-Infected Patients and Correlate with CD4+ T-Cell Counts”, authored by Nikolaos Sipsas, Petros P. Sfikakis, Athanasios Kontos and Theodore Kordossis, published in “Clinical and Diagnostic Laboratory Immunology”, May 2002, p. 558-561, Vol. 9, No. 3, it has been recently determined that the serum levels of this protein appear to be directly proportional to CD4+ T cell counts, making this protein a potentially ideal surrogate marker for determining the immune status of HIV/AIDS patients.

The device 10 is designed (i) to put routine immune status testing within the reach of far more HIV/AIDS patients, particularly in resource-scarce or resource-poor areas; (ii) to provide and make available an extremely affordable and easy to use rapid diagnostic test for CD4+ T cell levels; (iii) for the determination of CD4+ T cell levels that can be performed without any special instrumentation and which will require no highly skilled personnel, fresh water or electricity; (iv) for the determination of CD4+ T cell levels within 20 minutes while the patient is still present, so there is no need for call back, days or weeks later, to read or interpret test results; (v) to give clinicians the ability to quickly diagnose compromised immune status due to HIV infection while the patient is still in their office allowing the immediate start of ART, if necessary; (vi) to provide a cost effective means of monitoring the efficacy of ART and the patient's treatment adherence, even in the most remote of locations; (vii) for the testing of CD4+ T cell levels at a cost of approximately $5.00 versus the current cost of $25-$50 (estimated cost including reagents, equipment, sample transport, etc.); for testing that can become accessible to more patients in more settings, helping to optimize ART and the allocation of limited supplies of ART drugs to the patients who need them most; (viii) to allow for the determination of CD4+T cell levels not by measuring the cells themselves but by measuring CD4+ T cell equivalents, i.e., soluble CD40 ligand/CD 154, and where such determination is envisioned to become routine in all countries and provide timely access to anti-retroviral medication even in resource-poor countries; and (ix) to allow the determination of CD4 cell levels without the -need for lysis of such cells.

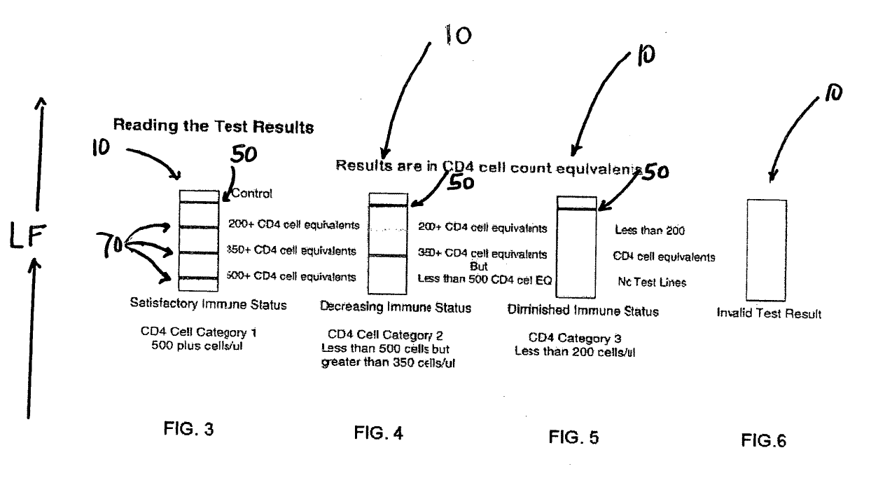

The process of using the semi-quantitative, immunochromatographic device 10 comprises the steps of placing a serum, plasma or whole blood sample at one end of the device 10. Thereafter, the sample migrates via lateral flow across the device 10 through an area containing antigen binding monoclonal or polyclonal antibody conjugated to colloidal gold particles, which binds the soluble CD40 ligand/CD 154 in the sample to form a complex. Thereafter, the complex moves via lateral flow over a unique combination of monoclonal and/or polyclonal antibodies immobilized on the device, which in turn bind the complex to generate a pink or purple band or bands on the device 10. The remaining complex continues to migrate to a control area on the device 10, which produces an additional band to show that the test has been performed correctly. Each pink or purple band appearing in the test area of the device 10 corresponds to a number of CD4+ T cell equivalents, which in turn can be correlated for example to one of three categories (see FIGS. 3, 4, 5 and 6): Satisfactory Immune Status, e.g., about 500 plus CD4+ T cells/μl; Decreasing Immune Status, e.g., less than about 500 plus CD4+ T cells/μl but greater than about 350 CD4+ T cells/μl; or Diminished Immune Status, e.g., less than about 200 CD4+ T cells/μl.

The semi-quantitative immunochromatographic device 10 comprises a support structure 20 having a top side 22, a bottom side 24, a proximate end 26, and a distal end 28. On said top side 22, at the proximate end 26 of said support structure 20, said semi-quantitative immunochromatographic device 10 further comprises a biological sample receiving area or sample pad 40; a conjugate pad/detector ligand area 60, immediately adjacent to, downstream from and communicatingly connected to said sample pad 40; a membrane 30 having a first end 32 and a second end 34 opposite to said first end 32, said membrane 30 being immediately adjacent to, downstream from and communicatingly connected to conjugate pad/detector ligand area 60; and a sink pad 90 located at the distal end 28 of the device 10 immediately adjacent to, downstream from and communicatingly connected to said membrane 30, such that all of the aforementioned components are capable of allowing and not impeding lateral flow, that is, flow in the direction of the arrow LF in FIG. 1.

The semi-quantitative immunochromatographic device 10 is essentially a composite of its aforementioned components. The steps of forming the composite device 10, comprise: placing said membrane 30 on said top side 22 of said support structure 20, such that said proximate end 26, and said distal end 28 extend beyond the outer perimeter of the area defined by the placement of said membrane 30 on said support structure 20; placing said conjugate pad/ligand detector 60 on top of the outer edge of first end 32 of said membrane 30 such that part of said conjugate pad/ligand detector area 60 lies partially on top of-and is supported by said membrane 30 and part of it extends upstream beyond said membrane and is partially supported by said support structure 20; further placing said sink pad 90 on top of the outer edge of said second end 34 of said membrane 30 such that part of said sink pad 90 lies on top of and is partially supported upstream by said membrane 30 and part of it extends beyond said membrane 30 and is partially supported by said distal end 28 of said support structure 20; and placing said biological sample receiving area or sample pad 40 at said proximate end 26 of said support structure 20 and over said conjugate pad/ligand detector 60 such that part of said sample pad 40 overlaps and is partially supported downstream by said conjugate pad/ligand detector 60 and partially by said proximate end 26 of said support structure 20.

As illustrated in FIG. 2, said support structure 20 can be shaped in the form of a strip. Alternatively, said support structure 20 can be provided in a wide variety of shapes or forms so long as the particular form permits the various functions described herein.

The support structure 20 can be formed from a number of different suitable materials, provided that the materials allow the aforementioned lateral flow functionality. For example, the materials can comprise vinyl with adhesive or polyester with adhesive, the adhesive being present to add cohesion to the remaining components of said device 10 and prevent said device 10 from falling apart.

The membrane 30 can be formed from a number of different suitable materials so long as such materials allow the aforementioned lateral flow functionality while remaining neutral and unreactive. For example, said membrane 30 can comprise glass fiber, cellulose ester, nylon, cross-linked dextran, etc. According to one embodiment, said membrane 30 comprises nitrocellulose.

As set forth herein above, said membrane 30 has a first end 32 and a second end 34. The edge of said second end 34 extends and fits under said sink pad 90 such that part of said sink pad 90 lies on top of and is partially supported upstream by and communicatingly connected to said membrane 30 and part of it extends beyond said membrane 30 and is partially supported by said distal end 28 of said support structure 20. The edge of said first end 32 extends and fits under said conjugate pad/detector ligand area 60, upstream therefrom and communicatingly connected thereto. Adjacent to said second end 34, but spaced from said sink pad 90, said membrane optionally further comprises a control line area 50. The control line area 50 defines an area which contains a control reagent. The control reagent contained within the control line area 50 is immobile, fixed upon said membrane 30. According to an illustrative non-limiting example, the control reagent is goat anti-mouse IgG.

The control reagent is capable of binding the conjugated detector ligand but is not specific for the soluble CD40 ligand/CD 154. Once it binds the complexed detector ligand, it immobilizes it and continues the lateral flow. Furthermore, a visible colored line appears so as to show that the device 10 is functioning properly.

In between the control line area 50 and the conjugate pad/detector ligand area 60, said membrane 30 further comprises at least one capture area or test line 70. Alternatively, said membrane 30 can further comprise a plurality of capture areas or test lines 70 (see FIGS. 3, 4 and 5). Each capture area or test line contains a capture reagent therein. The capture reagent is immobily and fixedly contained within said capture area or test line 70. The capture reagent comprises any mouse monoclonal and/or polyclonal anti-sCD40 ligand/CD 154. According to the illustrated embodiments, the capture areas or test lines 70 are distinct and separate from one another in the direction of lateral flow LF. However, it is comprehended by the present invention that one or more capture areas or test lines 70 can be combined and or otherwise merged. Additionally, location, shape, size and configuration of the capture areas/test lines 70 may also deviate from that of the illustrated embodiments.

The biological sample receiving area or sample pad 40 on the proximate end 26 of the device 10 acts as the repository of the patient or biological sample to be tested. It can be formed of cellulose, glass fiber and/or any other material that may also provide for the separation of plasma or serum from whole blood samples. While in the illustrated embodiment only one biological sample receiving area or sample pad 40 is shown located at the proximate end 26 of the device 10, it should be understood that the present invention can comprise a plurality of sample pads 40 on the proximate end 26 of the device 10. Moreover, the sample pad 40 can have locations which differ from that of the illustrated embodiment.

The conjugate pad/detector ligand area 60 comprises, optionally, a glass fiber pad, and a detector ligand that is contained within the glass fiber pad in a manner that renders it mobile. In other words, the detector ligand is capable of being carried out of the area 60 by the aforementioned lateral flow. According to an illustrative non-limiting example, a suitably concentrated solution or suspension of detector ligand is applied within area 60 and dried. The detector ligand preferably comprises mouse monoclonal and/or polyclonal anti-sCD40 ligand/CD 154 conjugated to colloidal gold particles. Alternatively, the detector ligand preferably comprises mouse monoclonal and/or polyclonal anti-sCD40 ligand/CD 154 conjugated to colloidal selenium particles; or mouse monoclonal and/or polyclonal anti-sCD40 ligand/CD 154 conjugated to colloidal charcoal particles; or mouse monoclonal and/or polyclonal anti-sCD40 ligand/CD 154 conjugated to colloidal latex particles or other suitable colored, fluorescent or magnetic micro particles.

When the detector ligand comes into contact with the soluble CD40 ligand/CD 154 in a biological sample being analyzed, it forms a complex that is also capable of being carried out of the area 60 by lateral flow. When the complex reaches said capture area(s)/test line(s) 70 on said membrane 30, the complex reacts with said capture reagent contained therein and becomes immobily bound to said capture reagent to produce a visible signal, as for example a pink or purple line. When no pink or purple line appears, such as shown in FIG. 6, this could be an indication of an invalid test result.

To the extent that the biological sample contains an amount of soluble CD40 ligand/CD 154 such that the capacity of the capture reagent contained in the capture area/test line 70 is exceeded, any such soluble CD40 ligand/CD 154, and complex formed thereby, continues to travel under the influence of the lateral flow, thereby reaching one or more optionally provided additional capture area/test lines 70.

Controlling the amount of capture reagent contained in the first capture area/test line 70 can be utilized to define a barrier beyond which an amount or concentration of soluble CD40 ligand/CD 154 contained in a biological sample may not pass. To the extent this minimum threshold value is exceeded, excess soluble CD40 ligand/CD 154 is free to travel under the lateral flow to one or more capture areas/test lines 70 which again establish increasing minimum threshold levels of soluble CD40 ligand/CD 154 under analysis. In this regard, the amount of capture reagent provided in each capture area can be the same amount, relative to one another. Alternatively, the amount of capture reagent contained in each capture area can progressively increase or decrease.

The presence of sufficient amount of soluble CD40 ligand/CD 154 and the complex formed from the CD40 ligand/CD 154 and detector ligand in the above-described one or more capture areas/test lines 70 is indicated by the generation of a detectable signal. This detectable signal can be generated in a number of different ways familiar to those skilled in the art. According to one example, the detector ligand can comprise a substance that is immediately and continuously visible to the naked eye. Thus, the mere physical presence of the complex formed between the soluble CD40 ligand/CD 154 and the detector ligand on the one or more capture areas/test lines 70 is sufficient to produce the desired detectable signal. Alternatively, the detector ligand can comprise a first reactant which becomes associated with the complex, formed with the soluble CD40 ligand/CD 154, but which is not visible. A second reactant can then be provided in the one or more capture areas/test lines 70 which upon combination and interaction with the first reactant produces a detectable signal.

In the illustrated embodiment, the area 60 containing the detector ligand and the biological sample receiving area 40 are illustrated as separate and distinct areas on the device 10. However, it is within the scope of the present invention that these two areas could be combined so as to define a single indistinct area of the device 10.

The device 10 could also be optionally provided with a housing (not shown). The housing can be formed of any suitable material. For example, the housing may be formed of a plastic material such as Mylar, polystyrene, ABS, etc. The housing at least partially encloses or surrounds the support structure 20. It may be provided with a sample pad window (not shown), a control line area window (not shown) and capture area(s)/test line(s) window(s) (not shown). According to the illustrated embodiments, the area containing the detector ligand is obscured from view of the user by the housing. It is of course comprehended that the area containing the detector ligand may be visible through the housing to the user as well. Moreover, the housing can be formed from a clear or translucent plastic material.

The housing can be optionally provided with indicia which identify various gradations of the concentration of the target CD40 ligand/CD 154 determined to be present in the biological sample by the device 10. As evident from the indicia illustrated in FIGS. 3, 4 and 5, it is possible to ascertain within a particular range of values the concentration of the CD4+ T cell equivalents, i.e., soluble CD40 ligand/CD 154 present in a biological sample, as described in further detail herein.

The sink pad 90 is made of any material that can act to absorb/stop the lateral flow and permits the lateral flow of the reagents and subsequently absorbs such reagents at the distal end of the test strip after migration 10.

An analysis performed according to the present invention comprises the following steps:

collecting an appropriate biological sample from an HIV-positive patient;

aspirating an appropriate sample volume of the biological sample using a sample pipette.

The biological sample can consist of whole blood, serum, or plasma;

depositing the aspirated biological sample to the biological sample receiving area or sample pad 40 of the device 10;

promoting lateral flow (LF) of the biological sample from the sample pad 40 into and onto the conjugate pad/detector ligand area 60, which contains the detector ligand, e.g., mouse monoclonal and/or polyclonal anti-sCD40 ligand/CD 154 coated on colloidal gold particles, or colloidal selenium particles, or colloidal charcoal particles or colloidal latex particles;

complexing the detector ligand by binding it to the soluble CD40 ligand/CD 154;

moving the complex, via lateral flow, from said conjugate pad/detector ligand area 60 into and onto at least one capture area/test line 70 immobilized on said membrane 30 and containing an immobile capture reagent configured to specifically bind with said complex;

reacting with said complex and immobile capture reagent on said capture area/test line 70 to become immobily bound to the capture reagent and produce a detectably visible signal, such as a pink or purple line; and

once a detectable visible signal is formed on said membrane 30, optionally promoting the lateral flow of whatever detector ligand is left uncomplexed in the biological sample from the capture area/test line 70 to a control line area 50 containing a control reagent which reacts with the uncomplexed detector ligand to produce another visible signal. In this region, the control line area serves as an internal procedural control and the detection of a signal in this area verifies that capillary flow has taken place and that the functional integrity of the device was maintained.

The results of several tests performed on HIV-infection blood samples using the immunochromatographic device of the present invention are provided below in Table I to show the efficacy and operability of the present invention to determine quickly and accurately a patient's immune status by determining the presence of, and quantifying, soluble CD40 ligand/CD 154 in a blood sample. The particular immunochromatographic device of the present invention used in the performance of these tests was constructed to provide a visible indication (in the form of a band) of soluble CD 40 ligand/CD 154 on the level of about 200 CD4+ T cells/μL.

TABLE I
TEST RESULTS

| | | | Immune- | Immune-STATUS Test Results | |
|---|---|---|---|---|---|
| | | | STATUS Rapid | Ranked as Positive (>200 CD4 | |
| | | | Test Type | EQ's) or Negative (<200 CD4 | |
| | | | Immune- | EQ's) | |
| | | | STATUS Quick | NOTE: | |
| Sample | CD4 | Viral | Screen | Negative = No Test Line | Date |
| Number | Count | Load | CD4 EQ 200 | Positive = Any visible Test Line | Run |
| 0005-041- | 1000 | | QS200 | Positive | Jul. 01, 2007 |
| 01472 | | | | | |
| 0006-041- | 225 | | QS200 | Negative | Jul. 01, 2007 |
| 01825 | | | | | |
| 0008-041- | 174 | | QS200 | Negative | Jul. 01, 2007 |
| 00640 | | | | | |
| 0008-214- | 528 | | QS200 | Positive | Jul. 01, 2007 |
| 02462 | | | | | |
| 0009-041- | 354 | | QS200 | Positive | Jul. 01, 2007 |
| 01357 | | | | | |
| 0006-041- | 994 | 63 | QS200 | Positive | Sep. 05, 2007 |
| 00754 | | | | | |
| 0008-041- | 599 | 188 | QS200 | Positive | Sep. 05, 2007 |
| 01001 | | | | | |
| 0008-214- | 200 | 5730 | QS200 | Positive | Sep. 05, 2007 |
| 02455 | | | | | |
| 0009-041- | 554 | <25 | QS200 | Positive | Sep. 05, 2007 |
| 01333 | | | | | |
| 0009-041- | 376 | <50 | QS200 | Positive | Sep. 05, 2007 |
| 01345 | | | | | |
| 0010-258- | 400 | 1640 | QS200 | Positive | Sep. 05, 2007 |
| 02458 | | | | | |
| 0006-041- | 402 | <50 | QS200 | Positive | Sep. 24, 2007 |
| 01826 | | | | | |
| 0009-096- | 451 | 800 | QS200 | Positive | Sep. 24, 2007 |
| 01781 | | | | | |
| 0009-214- | 501 | <50 | QS200 | Positive | Sep. 24, 2007 |
| 01609 | | | | | |
| 0010-214- | 456 | 15679 | QS200 | Positive | Sep. 24, 2007 |
| 00041 | | | | | |
| 0005-041- | 278 | 13211 | QS200 | Positive | Nov. 06, 2007 |
| 01454 | | | | | |
| 0005-041- | 301 | 6629 | QS200 | Positive | Nov. 06, 2007 |
| 01695 | | | | | |
| 0006-041- | 254 | 26801 | QS200 | Positive (very weak) | Nov. 06, 2007 |
| 00772 | | | | | |
| 0006-041- | 256 | <50 | QS200 | Positive (very weak) | Nov. 06, 2007 |
| 01524 | | | | | |
| 0007-041- | 205 | 70 | QS200 | Negative | Nov. 06, 2007 |
| 01775 | | | | | |
| 0008-041- | 226 | 4972 | QS200 | Negative | Nov. 06, 2007 |
| 00993 | | | | | |
| 0009-214- | 301 | 39652 | QS200 | Positive | Nov. 06, 2007 |
| 01940 | | | | | |
| 145719 | 1165 | N/A | QS200 | Positive | Oct. 08, 2007 |
| 145721 | 920 | N/A | QS200 | Positive | Oct. 08, 2007 |
| 145723 | 1012 | N/A | QS200 | Positive | Oct. 08, 2007 |
| 145725 | 578 | N/A | QS200 | Positive | Oct. 08, 2007 |
| 145726 | 641 | N/A | QS200 | Positive | Oct. 17, 2007 |
| 146106 | 923 | N/A | QS200 | Positive | Oct. 17, 2007 |
| 146108 | 880 | N/A | QS200 | Positive | Oct. 17, 2007 |
| IN 01 | 457 | N/A | QS200 | Positive | Dec. 06, 2007 |
| IN 02 | 142 | N/A | QS200 | Positive (very weak)* | Dec. 06, 2007 |
| IN 03 | 304 | N/A | QS200 | Positive | Dec. 06, 2007 |
| IN04 | 604 | N/A | QS200 | Positive | Dec. 20, 2007 |
| IN05 | 287 | N/A | QS200 | Negative | Dec. 20, 2007 |
| IN06 | 99 | N/A | QS200 | Positive (very weak)* | Dec. 20, 2007 |
| IN07 | 214 | N/A | QS200 | Positive (very faint) | Dec. 20, 2007 |
| IN08 | 537 | N/A | QS200 | Positive | Dec. 20, 2007 |
| IN09 | 634 | N/A | QS200 | Positive | Dec. 20, 2007 |
| IN10 | 225 | N/A | QS200 | Positive (weak line) | Dec. 20, 2007 |
| IN11 | 32 | N/A | QS200 | Positive* | Dec. 20, 2007 |
| IN12 | 171 | N/A | QS200 | Positive (very faint line)* | Dec. 20, 2007 |
| IN13 | 193 | N/A | QS200 | Positive (very faint line)* | Dec. 20, 2007 |
| IN14 | 395 | N/A | QS200 | Positive | Dec. 20, 2007 |
| IN15 | 384 | N/A | QS200 | Positive | Dec. 20, 2007 |
| IN16 | 103 | N/A | QS200 | Negative | Dec. 20, 2007 |
| IN17 | 639 | N/A | QS200 | Positive | Dec. 20, 2007 |
| SA1 | 133 | N/A | QS200 | Positive* | Mar. 10, 2008 |
| SA2 | 176 | N/A | QS200 | Positive | Mar. 10, 2008 |
| SA3 | 211 | N/A | QS200 | Positive | Mar. 10, 2008 |
| SA4 | 222 | N/A | QS200 | Positive | Mar. 10, 2008 |

TABLE I - COMMENTS
*Results marked with an asterisk (*) are discordant.
All results at 200 +/− 25 CD4 cells were considered positive based on the tests low end cutoff
N = 50 (As of March 2008)
Correlation to Flow Cytometry = 89.1%

Results for the rapid test performed with the immunochromatographic device of the present invention are reported in Table I as either “positive” when a colored band of any intensity is seen in the test area of the device or “negative” when no colored band is visible in the test area. As the amount of CD4 equivalent proteins approaches the dynamic cutoff for the test which is approximately 200±25 CD4 cells, the colored band in the test area of the device will become lighter and lighter until it disappears at approximately 200±25 CD4 cells.

The immunochromatographic device of the present invention provides a one step screening test for the semi-quantitative determination of immune status in HIV/AIDS patients via detection of specific proteins that directly correlate to CD4 counts (CD4 equivalents). To reiterate, AIDS is characterized by changes in the amount of T-cell lymphocytes. The virus, in infected individuals, causes a depletion of the T-helper cells, which are a sub population of T-cells. This leaves patients susceptible to opportunistic infections and potential malignancies. The presence of the virus itself causes the immune system to deteriorate as AIDS progresses. In normal and immune suppressed individuals, there are specific proteins that are detectable that correlate to CD4 counts and which can be detected by the device and method of the present invention, as evidenced by the test results shown in Table I. Tests such as CD4 counts are among the most widely used method for determining the immune status of HIV/AIDS infected patients and to establish the efficacy and/or timing of the start of ART (anti-retroviral therapy).

The present invention provides a rapid membrane based screening test to detect the presence of specific proteins that correlate to CD4 counts. This test is the newest generation lateral flow immunochromatographic type assay. These are among the simplest and easiest to use POC (point of care) assays requiring no instrumentation or highly skilled individuals to perform. Test using the device of the present invention can be performed using fresh plasma or serum samples. The test employs the use of an antigen binding monoclonal antibody conjugated to a colloidal gold particle and a unique combination of monoclonal antibodies immobilized on the membrane.

As described previously, once the sample is added to the test device, the mixture passes through the antigen binding/gold complex, which then binds the specific target protein in the sample. As this complex passes over the immobilized antibodies on the membrane, if any specific CD4 equivalent proteins are present, the antibodies capture them in turn. This produces a pink/purple band or bands in a particular area of the test device. The remaining complex continues to migrate to a control area on the test device and produces a pink/purple band in this control area. This control band indicates that the test has been performed properly.

As a result of the components of the inventive device described herein and its various embodiments disclosed herein above and the way they cooperatingly function, it is clear that they achieve all of the objectives set forth herein above including: (i) putting routine immune status testing within the reach of far more HIV patients, particularly in resource-scarce or resource-poor areas; (ii) providing and making available an extremely affordable and easy to use rapid diagnostic test for CD4+ T cell levels; (iii) the determination of CD4+ T cell levels that can be performed without any special instrumentation and which will require no highly skilled personnel, fresh water or electricity; (iv) the determination of CD4+ T cell levels within 20 minutes while the patient is still present, so there is no need for call back, days later, to read test results; (v) giving clinicians the ability to quickly diagnose compromised immune status due to HIV while the patient is still in their office and immediately begin ART, if necessary; (vi) providing a cost effective means of monitoring the efficacy of ART and the patient's treatment adherence, even in the most remote of locations; (vii) the testing of CD4 cell levels at a projected cost of approximately $5.00 versus the current cost of $25-$50 (estimated cost including reagents, equipment, sample transport, etc.); (viii) allowing for the determination of CD4+T cell levels not by measuring the cells themselves but by measuring CD4+ T cell equivalents, i.e., soluble CD40 ligand; and (ix) allowing the determination of CD4 cell levels without the lysis of such cells.

While particular embodiments of the invention have been illustrated and described in detail herein, they are provided by way of illustration only and should not be construed to limit the invention. Since certain changes may be made without departing from the scope of the present invention, it is intended that all matter contained in the above description or shown in the accompanying drawings be interpreted as illustrative and not in a literal sense. Practitioners of the art will realize that the sequence of steps and the embodiments depicted in the figures can be altered without departing from the scope of the present invention and that the illustrations contained herein are singular examples of a multitude of possible depictions of the present invention.

What is claimed is:

1. A device for the detection of an HIV/AIDS immune status CD4+ T cell equivalent, which comprises:
one or more support materials capable of providing lateral flow;
a first area situated on at least one of the one or more support materials for receiving a biological sample containing a target analyte, the analyte being a CD4+ T cell equivalent;
a second area situated on at least one of the one or more support materials having a movably contained detector ligand conjugate, wherein the detector ligand is capable of forming a mobile complex with the CD4+ T cell equivalent; and
at least one capture area situated on at least one of the one or more support materials having a predetermined amount of an immobile capture reagent, the immobile capture reagent capable of specifically binding to the mobile complex formed by the CD4+ T cell equivalent protein and the detector ligand and providing a visible signal.

2. A device as defined by claim 1, wherein the CD4+ T cell equivalent is soluble CD40 ligand/CD 154.

3. A device as defined by claim 2, wherein the detector ligand of the second area includes at least one of mouse monoclonal and/or polyclonal anti-sCD40 ligand/CD 154 conjugated to colloidal gold particles; mouse monoclonal and/or polyclonal anti-sCD40 ligand/CD 154 conjugated to colloidal selenium particles; mouse monoclonal and/or polyclonal anti-sCD40 ligand/CD 154 conjugated to colloidal charcoal particles; and mouse monoclonal and/or polyclonal anti-sCD40 ligand/CD 154 conjugated to colloidal latex particles.

4. A device as defined by claim 2, wherein the immobile capture reagent situated on at least one capture area includes a mouse monoclonal and/or polyclonal anti-sCD40 ligand/CD 154.

5. A device as defined by claim 1, which further comprises:
a control area situated on at least one of the one or more support materials the control area including an immobile control reagent providing a visible indication and which is capable of binding the conjugated detector ligand but does not recognize or bind to the CD4+ T cell equivalent, whereupon the control reagent binds the conjugated detector ligand and changes color so as to show that the device is working properly.

6. A device as defined by claim 5, wherein the control reagent includes goat anti-mouse IgG.

7. A device as defined by claim 2, wherein the detector ligand includes a first reactant which becomes associated with the complex formed with the soluble CD40 ligand/CD 154, and a second reactant situated on the at least one capture area which upon binding and interaction with the first reactant produces a detectable signal.

8. A method of using a semi-quantitative, immunochromatographic device, which comprises the steps of:
placing a serum, plasma or whole blood sample at one end of the device on a first area on the device that contains a target analyte, the target analyte being a CD4+ T cell equivalent;
moving the sample, via lateral flow, to a second area on the device that contains a conjugated detector ligand capable of forming a mobile complex with the CD4+ T cell equivalent; and
moving the complex, via lateral flow, to at least one capture area on the device having a predetermined amount of an immobile capture reagent capable of binding to the mobile complex formed by the CD4+ T cell equivalent and the conjugated detector ligand and providing a visible indication.

9. A method as defined by claim 8, wherein the CD4+ T cell equivalent is soluble CD40 ligand/CD 154.

10. A method as defined by claim 8, which further comprises the step of:
moving the complex, via lateral flow, over the at least one capture area having a predetermined amount of an immobile capture reagent, allowing the remaining complex to continue to flow to a control area on the device having a control reagent situated thereon which produces a visible indication that the method has been performed correctly.

11. A method as defined by claim 9, wherein the detector ligand is at least one mouse monoclonal and/or polyclonal anti-sCD40 ligand/CD 154 conjugated to colloidal gold particles; mouse monoclonal and/or polyclonal anti-sCD40 ligand/CD 154 conjugated to colloidal selenium particles; mouse monoclonal and/or polyclonal anti-sCD40 ligand/CD 154 conjugated to colloidal charcoal particles; and mouse monoclonal and/or polyclonal anti-sCD40 ligand/CD 154 conjugated to colloidal latex particles.

12. A method as defined by claim 9, wherein the capture reagent includes a mouse monoclonal and/or polyclonal anti-sCD40 ligand/CD 154.

13. A method of detecting or quantifying the presence of CD4+ T cells for the detection of HIV/AIDS, which comprises the step of detecting or quantifying the presence of soluble CD40 ligand/CD 154.
